(19) United States
(12) Patent Application Publication
(10) Pub. No.: US 2010/0280370 A1
(43) Pub. Date: Nov. 4, 2010

(54) Syringe plunger

(76) Inventor: David Namey, JR., New Kensington, PA (US)

Correspondence Address: FLOYD B. CAROTHERS;CAROTHERS AND CAROTHERS, 445 FORT PITT BOULEVARD, SUITE 200, PITTSBURGH, PA, 15219, US

(21) Appl. No.: 12/804,064

(22) Filed: Jul. 12, 2010

Related U.S. Application Data
(62) Division of application No. 12/383,694, filed on Mar. 26, 2009, now abandoned.

(51) Int. Cl. A61M 5/315; A61B 6/00

(52) U.S. Cl. 600/432

(57) ABSTRACT

A syringe plunger is manufactured by injecting polypropylene with filler into a mold for forming the hard core of the plunger, and immediately thereafter co-injecting a thermoplastic elastomer into a cover mold positioned at the forward end of the plunger mold cavity to thereby form a soft exterior cover over the forward end of the hard plunger core with a secure chemical bond.

### CROSS REFERENCE

This application is a division of U.S. application Ser. No. 12/383,694, filed Mar. 26, 2009 for Syringe Plunger and Method of Manufacture.

### BACKGROUND OF THE INVENTION

The present invention relates generally to plungers for use in syringes to inject fluid into a patient and to methods of fabrication thereof.

The syringe plungers of the present invention pertain particularly to injector-actuated syringes and powered injectors for use in medical procedures, such as, angiography, computed tomography, ultrasound and magnet resonance imaging (MRI).

To date, the most effective and common method of manufacturing such syringe plungers is to first form a hard plastic core in a first mold, and then subsequently the distal portion of the hard plastic core is overmolded with a soft rubber plunger cover in a second mold. U.S. Pat. No. 5,902,276 by the same inventor herein, the disclosure of which is incorporated herein by reference, discloses that overmolding can occur in the second mold while the core is still cooling, or not completely cured, to effect molecular bonding of the rubber of the cover with the as-yet uncured plastic of the core. A similar process is disclosed in WO 2004/035289.

Yet another overmolding process is disclosed in US Patent Application Publication No. 2007/0219508, the disclosure of which is incorporated herein by reference. In this overmolding process, the elastomeric sealing portion of the syringe plunger is injection molded first and then the central hard core is injection overmolded thereon in a second mold. In another embodiment, the central portion is injection molded first and the sealing portion is injection overmolded thereon in a second mold. The reference further discloses that material for the sealing portion and the material for the central portion can be selected so that molecular bonding occurs between the sealing portion and the central core portion.

However, it is recognized in all of these prior art references that such molecular bonding may not in and of itself be considered to provide a sufficient and secure bond, and accordingly, these references further suggest the addition of mechanical bonding wherein parts of the hard central core and parts of the elastomer sealing portion are interconnected mechanically to provide a better interconnecting bond between the two.

It is a primary object of the present invention to provide a syringe plunger and method of manufacture thereof wherein superior bonding between the plunger elastomer sealing portion and the central hard core portion is provided without the requirement of any mechanical bond and wherein bonding is provided between the elastomer sealing cover portion and the hard core which is greater than a molecular bond heretofore provided in the prior art. This would provide a syringe plunger which is less expensive to manufacture and which may be manufactured at a faster rate than with the prior art overmolding techniques.

### SUMMARY OF THE INVENTION

The syringe plunger of the present invention is comprised of a hard core that is composed of polypropylene and filler, and the soft exterior cover of thermoplastic elastomer is molded with a forward end of the core by co-injection during molding of the core. Accordingly, the soft exterior is chemically bonded, thereby fusing the cover and core together, as opposed to being merely molecularly bonded, to the hard core as a consequence of the co-injection process of the selected core and cover compositions. No mechanical bonding is required and the chemical bond is such that the soft exterior cover and the hard plastic core are integrated as one, as opposed to a mere molecular bonding of the moldover process of the prior art which provides a relatively strong bond, but not sufficient to be considered an inseparable bond.

The overmolding processes of the prior art generally select a thermoplastic elastomer or a thermoplastic polyurethane for the sealing portion, and acrylonitrile butadiene styrene, polycarbonate or copolymers thereof for the hard core. Polypropylene is also mentioned as a possibility. However, as a practicality, polypropylene is not suitable for use in manufacturing the hard core in and of itself as it provides a hard core which is not sufficiently strong. However, the present inventor has discovered that polypropylene may be used for this purpose with a filler, such as, glass fiber, mica, calcium carbonate or talc, or any combination thereof, as the core material. Adequate strength is thus provided in the central plunger core, and when this is used in combination by co-injection during molding with the soft exterior cover being selected as a thermoplastic elastomeric material, such as, SANTOPRENE®, ATPE available, for example from Advanced Elastomer Systems, L.P. of Akron, Ohio, and extremely strong chemical bonding is obtained between the elastomer soft exterior cover and the hard core and they are fused together.

In consequence, the manufacturing process for the syringe plunger of the present invention is in reality not an overmolding process as are all of the aforementioned prior art methods. Instead, the selected materials for the soft exterior cover and also for the hard core are simultaneously co-injected into the mold on a closely timed arrangement. In co-injection the core is injected into its mold and immediately rotated 180° to a second position to register with a different front head mold cavity for the soft cover. While the soft cover is being injected, another core is simultaneously being injected, and the process continues.

Instead of molding the plunger stem as part of the plunger core, considerable material is saved by making the hard core with a hollow interior cavity which is open to the rearward end of the core. The molded hard plastic plunger stem is then separately molded and dimensioned to fit and secure in this cavity with a forced fit. The stem also may have hollow portions to once again save material.

### BRIEF DESCRIPTION OF THE DRAWINGS

Other objects and advantages appear hereinafter in the following description and claims. The accompanying drawings show, for the purpose of exemplification, without limiting the scope of the invention or the appended claims, certain practical embodiments of the present invention wherein:

FIG. 1 is a perspective view of an embodiment of the plunger of the present invention;

FIG. 2 is a rear end view of the plunger shown in FIG. 1;

FIG. 3 is a perspective cutaway view of the plunger of FIG. 1, as seen along section line III-III of FIG. 2;

FIG. 4 is a view in side elevation of the plunger of FIG. 1, illustrating the hard core of the plunger of the present invention without the application of the soft exterior cover;

FIG. 5 is a perspective view of the embodiment illustrated in FIG. 1, illustrating the plunger with a plunger stem attached;

FIG. 6 is a view in side elevation of the plunger stem shown in FIG. 5 prior to insertion into the plunger; and

FIG. 7 is a front end view of the plunger stem illustrated in FIG. 6.

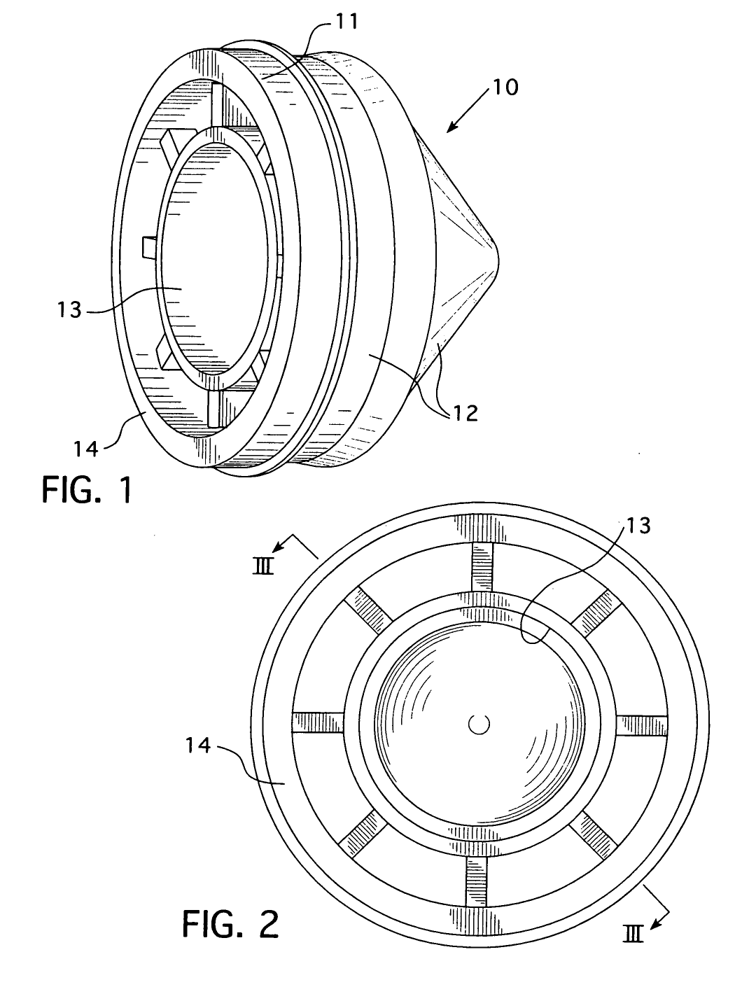

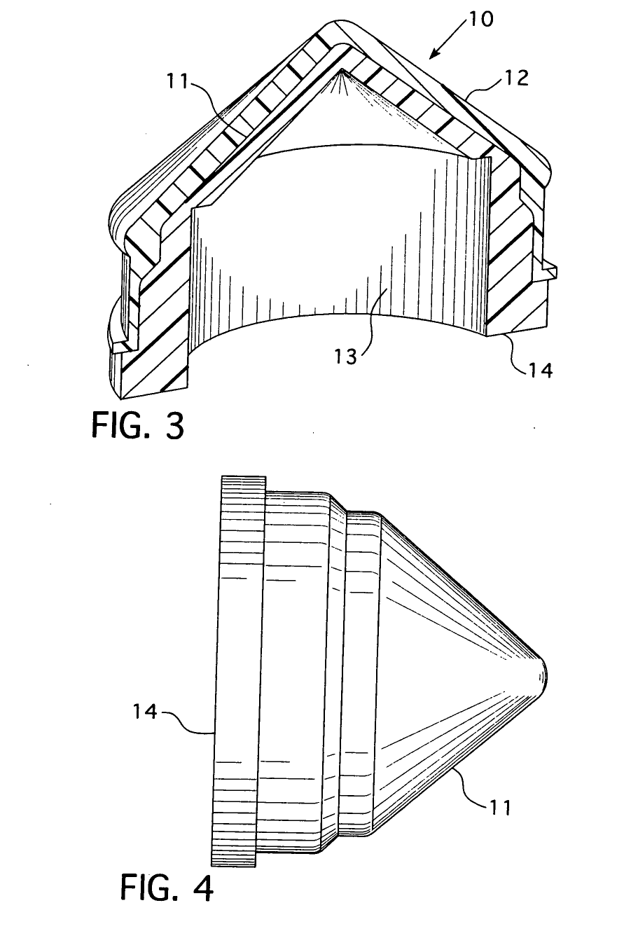

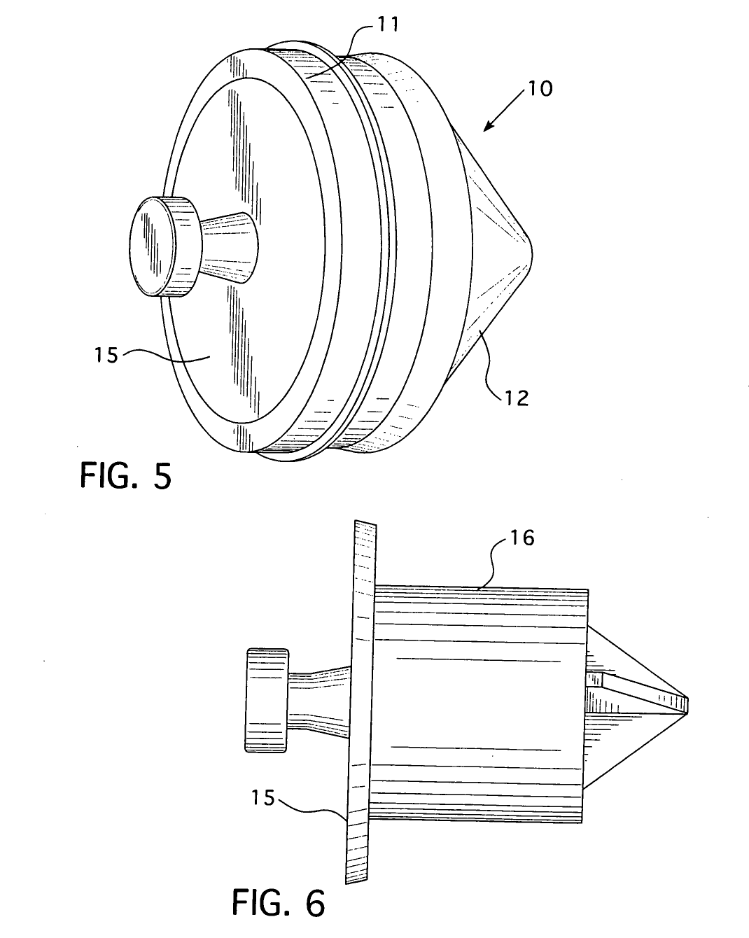

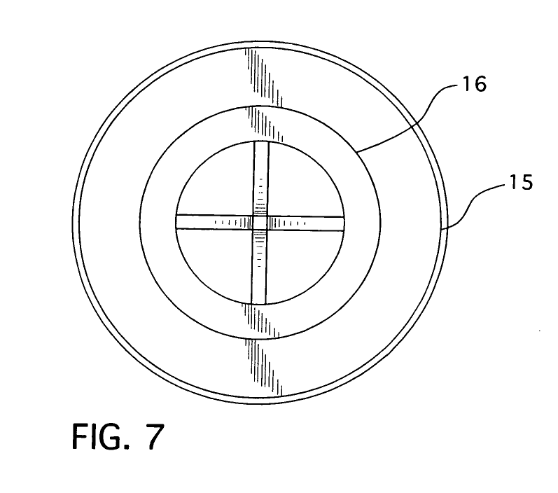

### DETAILED DESCRIPTION OF A PREFERRED EMBODIMENT

Referring to the drawings, the syringe plunger 10 of the present invention is composed of a hard core 11 and a soft exterior cover 12. The hard core 11 is composed of polypropylene with filler. The filler may be selected of any one or more of glass fiber, mica, calcium carbonate, or talc, or any equivalent filler or combination thereof. The soft exterior cover is composed of a thermoplastic elastomer, such as SANTOPRENE®.

The hard core material and the soft exterior cover material are co-injected into a mold (not shown) during the molding process previously described for the core/exterior cover molding process, whereby the soft exterior is chemically bonded and thereby fused, as opposed to being merely molecularly bonded, to the hard core as a consequence of the co-injection process of the specific core and cover compositions. In the prior art overmolding processes, the core must be removed from one mold and transferred to a second mold for overmolding of the soft exterior. Thus, excessive curing of the core occurs and the process is therefore not suitable for the present invention.

The core 11 is provided with a hollow interior cavity 13 which is open to the rearward end 14 of the core 11. This saves material and further provides a means for securing hard plastic plunger stem 15 to be inserted and secured. Plunger stem 15 is dimensioned at its cylindrical exterior 16 to fit within the annular hollow interior 13 of core 11 with a strong force fit until it is completely seated as illustrated in FIG. 5. This fit is sufficiently strong whereby the stem exterior 16 does not cause undo expansion of the plunger 10, yet prevents accidental removal or separation of the plunger stem 15 from the core 11.

I claim:

1. A syringe plunger comprising:
a hard core composed of polypropylene and filler; and
a soft exterior cover of thermoplastic elastomer molded in contact with a forward end of said core by co-injection during molding of said core, whereby said soft exterior is chemically bonded, as opposed to a mere molecular bond, to said hard core as a consequence of said co-injection process of said core and cover compositions.

2. The syringe plunger of claim 1, wherein said filler consists of at least one of glass fiber, mica, calcium carbonate or talc.

3. The syringe plunger of claim 1, wherein said hard core has a hollow interior cavity which is open to a rearward end of said core and no exterior cavities are provided for mechanical bonding with said elastomer.

4. The syringe plunger of claim 3, including a molded hard plastic plunger stem dimensioned to fit and secure in said cavity with a force fit.
